(19) United States
(12) Patent Application Publication
(10) Pub. No.: US 2021/0401700 A1
(43) Pub. Date: Dec. 30, 2021

(54) Total Body Moisture

(71) Applicant: Avis Garrett Burrell, Ocoee, FL (US)

(72) Inventor: Avis Garrett Burrell, Ocoee, FL (US)

(21) Appl. No.: 17/362,425

(22) Filed: Jun. 29, 2021

Related U.S. Application Data
(60) Provisional application No. 63/045,807, filed on Jun. 29, 2020.

(51) Int. Cl. A61K 8/36; A61K 8/67; A61Q 19/00; A61Q 17/04; A61Q 19/08; A61Q 17/00; A61Q 7/00; A61Q 19/02; A61K 8/92

(52) U.S. Cl. CPC A61K 8/361; A61K 8/678; A61K 8/671; A61Q 19/007; A61K 8/922; A61Q 19/08; A61Q 17/005; A61Q 7/00; A61Q 19/02; A61Q 17/04

(57) ABSTRACT

The invention is Total Body Moisture. The Butter is used for conditioning the scalp and hair promoting hair growth and moisturizing for natural, permed, or relaxed hair as well as healing the skin all over the entire human body. It can be used for moisturizing after bath or shower, protecting skin from some UV rays, healing minor cuts, bums, and abrasions. It also can be used to soothe itching skin from the effects of eczema or psoriasis. TBM is also used to decrease wrinkles, fine lines, and minor acne on the skin surface. The main base oil of TBM is pure African Shea Butter in any shade and variation. Carrier oils such as Coconut oil, jojoba oil, black castor oil, argan oil, hemp seed oil, wheat germ oil, almond oil, avocado oil, and grapeseed oil are used to add to the clinical benefits of the pure African shea butter base.

### BACKGROUND

My son was born with severe ectopic dermatitis. He suffered greatly during his infancy with food allergies that brought on the skin issues. I searched to find a natural remedy to help soothe his itchy painful cracked dry skin. I researched all-natural oils and essential oils and with the same healing properties and decided on a virgin pure unrefined African Shea Butter with natural carrier oils and fragrance oils. I added love to the mix and created Total Body Moisture. After using on my son's skin, we realized that the moisturizing butter mixture can also be used to moisturize and repair/heal the hair, nails, feet, hands, nails, lips, elbows, knees, dry heels, beards, all skin surfaces, and scalp helping to reduce blemishes on the skin, and treat minor burns, scrapes and abrasions. TBM also may reduce fine lines and wrinkles. Total Body Moisture can be used on all skin and hair types.

### SUMMARY

The following is intended to be a summary of the invention and is not intended to limit the scope of the invention. I ordered pure unrefined African Shea butter and pure virgin unrefined coconut oil online. I melted the shea butter into liquid form and combined all essential oil ingredients essential natural oil fragrances together with a mixer until emulsified to a creamy pourable consistency. I found the perfect blend, smell, color, texture. I tried it on my son's whole body for his eczema. I was looking for something to soothe his skin during outbreaks. It worked successfully as a healing moisturizer. I also used the mixture on my hair, nails, feet, elbows, and all over my entire body as a moisturizer. I also used the mixture on my hair and noticed several positive changes in the texture and moisture levels.

### DESCRIPTION

Four pounds of Raw Virgin African Shea Butter of any kind in any form in all shade variations is the base oil melted with indirect heat which contains the same properties as the oils from human sebaceous glands. TBM is made by melting down by indirect heat pure unrefined 100% pure African shea butter of any kind of any form of any variation. Add ⅛ cup natural pure Lavender oil or ⅛ cup natural pure peppermint oil or ½ teaspoon of natural pure vanilla oil or vanilla bean oil, or ⅛ cup of lemongrass oil. (Depending on the desired flavors which are Lavender-Vanilla, Peppermint, and Lemon Zest), one cup pure virgin coconut oil, ¼ cup 100% jojoba oil, ¼ cup all-natural tea tree oil, ¼ cup natural sweet almond oil, 100% pure avocado oil, !00% natural hemp seed oil, ⅛ cup pure argan oil, and 100% ¼ pure Jamaican black castor oil, ¼ pure vegetable glycerin, ¼ cup pure wheat germ oil to the melted pure unrefined African shea butter. This is to be mixed in all ratios per the ingredients with any industrial professional mixer until mixture is smooth, thick, and pourable in any consistency in any physical state. Put the mixture in the freezer for 5 minutes then remove from freezer and remix twenty minutes. Remove from freezer and mix on medium-high with a five-speed mixer until the liquid becomes a whipped solid material. Pour the whipped butter in a 12 oz plastic jar, place plastic lid top on jar, and put in the freezer for 10 mins or any amount of time for the desired consistency to set. Total Body Moisture aids in replenishing the skin and hair. The shea butter combined with the other oils, helps the skin become soft and smooth. It moisturizes and gives a shine to the hair and beard. Turn those dry, cracked, ashy feet into a smooth youthful look again. TBM also aids in the healing of eczema, minor scrapes, cuts, and burns. After every shower apply TBM all over your body, or more often as needed. A pea size amount will moisturize both hands and face. The product is packaged in a 12 oz plastic jar. The butter can be applied to wet or dry skin and wet or dry hair.

1. TBM will highly moisturize and heal the skin on entire body.

2. TBM has antioxidant and anti-inflammatory properties.

3. TBM sooths itchy skin and scalp.

4. TBM is a natural UV Blocker.

5. TBM may reduce fine lines and wrinkles.

6. TBM will add moisture to relaxed and natural hair.

7. TBM soothes and heals damaged skin due to eczema.

8. TBM reduce blemishes, dark spots, discolorations, stretch marks.

9. TBM does not clog pores.

10. TBM improves the skin's elasticity and provides deep hydration.

11. TBM promotes hair growth and heals scalp.

12. TBM promotes cell regeneration and oil balance on the skin, scalp, and hair.

13. TBM helps heal minor cuts, burns, and insect bites.

14. TBM helps w arthritis.

15. TBM has antibacterial and antifungal properties.

16. TBM contains vitamins E, A, and F

17. TBM heals dry cracked cuticles.

18. TBM heals and conditions diabetic skin and psoriasis.

19. TBM conditions skin, scalp, and hair.

20. TBM balances the natural oils on the skin's surface.
